(19) United States
(12) Patent Application Publication
(10) Pub. No.: US 2020/0299328 A1
(43) Pub. Date: Sep. 24, 2020

(54) POLYPEPTIDE WITH FUNCTION OF INHIBITING ABETA 42 PROTEIN AGGREGATION AND USE THEREOF, AND GENE ENCODING THE POLYPEPTIDE

(71) Applicant: SOUTH CHINA UNIVERSITY OF TECHNOLOGY, Guangdong (CN)

(72) Inventors: Jiaoyan REN, Guangdong (CN); Min WANG, Guangdong (CN)

(73) Assignee: SOUTH CHINA UNIVERSITY OF TECHNOLOGY, Guangdong (CN)

(21) Appl. No.: 16/627,763

(22) PCT Filed: Oct. 31, 2018
(86) PCT No.: PCT/CN2018/113238

§ 371 Date: Dec. 31, 2019

(30) Foreign Application Priority Data
May 24, 2018 (CN) 201810504181.X

(51) Int. Cl. C07K 7/06; A23L 33/18; A61P 25/28

(52) U.S. Cl. CPC C07K 7/06; A61K 38/00; A61P 25/28; A23L 33/18

(57) ABSTRACT

The invention discloses a polypeptide with a function of inhibiting Aβ42 protein aggregation and use thereof, and a gene encoding the polypeptide. The polypeptide with the function of inhibiting Aβ42 protein aggregation is called PW-5, and has an amino acid sequence of Pro-Pro-Lys-Asn-Trp. The polypeptide with the function of inhibiting Aβ42 protein aggregation according to the present invention can effectively inhibit aggregation and deposition of Aβ42 protein, so as to effectively improve memory ability of a body; and can be applied to preparation of a drug or a food with inhibiting Aβ42 protein aggregation function or can be applied to preparation of a drug, a health product or a food for preventing or treating Alzheimer's disease, so that preventing and treating situations of the Alzheimer's disease can be effectively improve.

### BACKGROUND OF THE INVENTION

### 1. Technical Field

The present invention relates to the technical field of polypeptide, and more particularly, to a polypeptide with a function of improving memory and use thereof.

### 2. Background Art

Commonly known as dementia, Alzheimer's disease (AD) is an age-related neurodegenerative disease characterized by progressive dementia, which is the most common form of dementia. As a hot spot in the research of neurological diseases at home and abroad, the treatment on a characteristic pathological feature—senile plaque of AD may be used as a very useful target spot for study. Studies showed that a large amount of amyloid β-peptide was accumulated in the brains of AD patients, which as 100 times to 1000 times higher than normal people. The main component of the senile plaque is amyloid β-protein (Aβ), while the formation of Aβ goes through β-APP gene-APP mRNA-β-APP forming-Aβ-Aβ deposition, and the deposition mainly presents in two forms of Aβ40 and Aβ42, but Aβ42 with a low content is more important. According to some early-onset hereditary AD, the level of Aβ1-42 in patients' serum is increased, and Aβ1-42 is a key component in vascular injury of AD. Aβ42 is a heterogeneous polypeptide containing 42 amino acid residues, which is an insoluble protein that can spontaneously and rapidly aggregate to form amyloid protein microscopy, and then becomes a seed for Aβ aggregation and subsequent deposition. Changes in a gene level can produce Aβ molecules with different fragment sizes, thus affecting an aggregation mode or a formation speed of the senile plaque. In the AD gene research, mutations are detected at some sites of some genes (such as APP, PSEN and PLD3), which can produce abnormal aggregation of the amyloid β-protein.

At present, animal models and in-vitro cell models are mostly used in the research of target point, i.e. senile plaque, of pathogenesis, development and treatment of AD. Although the animal models used in AD have their own characteristics, the animal models have their own shortcomings, high costs, and large individual differences and so on. Therefore, the animal models cannot be widely applied to the research on the pathogenesis of AD and related drug therapy. In-vitro experiments attract much attention due to the advantages of low costs, capability of preliminary screening of a large number of related substances for the treatment of AD, research time saving, etc. In addition, an Aβ polypeptide is mostly used in the existing in-vitro experiments of AD senile plaque to incubate neuron cells, resulting in the occurrence of an Aβ aggregation phenomenon. The use of such method is limited by the high synthesis cost of high-purity AP peptide, the instability of experimental results due to the inability of accurately quantifying the amount of Aβ entering cells in external incubation, and other reasons. In recent years, a large number of applications of DNA recombination technology in the biological field provide a new research idea for the research of AD senile plaque—an in-vitro cell model of abnormal protein aggregation. An amino acid sequence of Aβ1-42 is integrated into a cell gene, wherein the gene at the 22^{nd} site can be over-expressed by induction with an inducer after mutation treatment, so as to form specific protein aggregation.

A peptide is a structural and functional fragment forming a protein, and a large number of researches showed that the peptide with various biological functions had become a hot spot of research in the world. A biological active peptide mainly refers to a peptide compound that is beneficial to a vital movement of a living organism and has a physiological effect, which comes from a wide range of sources, can be derived from various living organisms, and can also be obtained by artificial synthesis or a bioengineering method. The active peptide has the advantages of active absorption, fast absorption speed, complete absorption and low consumption, and the physiological function of the active peptide is superior to an amino acid and a protein. Nowadays, the research on the biological active peptide is increasingly active at home and abroad, and many polypeptides with biological activity have been widely applied in practice as a diagnostic reagent, a drug, a vaccine, a functional food and so on.

Polypeptide is often prepared by an enzymatic hydrolytic method. However, obtaining the biological active polypeptide after protease hydrolysis, separation and purification takes a long time and high costs with a low yield, and meanwhile, a quality of the polypeptide obtained is difficult to be effectively controlled, thus restricting the large-scale production and application of the biological active peptide. This gave birth to a method for preparing the polypeptide by chemical synthesis, and at present, a solid-phase synthesis method is mostly used in the chemical synthesis of the polypeptide. The polypeptide synthesized by artificial chemistry has many advantages of high purity, low costs, less time required, controllable yield, etc. Moreover, with the continuous development of science and technology, and the continuous improvement of research technology, more polypeptides with biological activity will be synthesized and discovered.

In the present in-vitro model for functional application of a polypeptide substance capable of improving learning and memory ability, an in-vitro cell model stably transfecting an Aβ42 protein by genetic engineering is not found to verify the efficacy. However, the in-vitro model has unique advantage in visualization, and can also be quantitatively analyzed. In addition, the polypeptide prepared by artificial synthesis has a high purity, which effectively reduces the occurrence of a heat source problem, and has a very broad development prospect in the prevention or treatment of neurodegenerative diseases such as Alzheimer's disease.

### SUMMARY OF THE INVENTION

An objective of the present invention is to provide a polypeptide with a function of inhibiting Aβ42 protein aggregation, aiming at the defects in the prior art. The polypeptide can resist aggregation and deposition of an Aβ42 protein, has an effect of improving memory ability, and can be applied to preparation of a food, a health product and a drug for preventing or treating Alzheimer's disease.

An objective of the present invention is further to provide a gene encoding the polypeptide with the function of inhibiting Aβ42 protein aggregation.

Another objective of the present invention is further to provide use of the polypeptide with the function of inhibiting Aβ42 protein aggregation.

The objectives of the present invention are achieved by the following technical solutions.

A polypeptide with a function of inhibiting Aβ42 protein aggregation, namely a pentapeptide, called PW-5, has an amino acid sequence of Pro-Pro-Lys-Asn-Trp, as shown in SEQ ID No:1 in the sequence listing;
wherein Pro is an amino acid corresponding to a residue of proline, Lys is an amino acid corresponding to a residue of lysine, Asn is an amino acid corresponding to a residue of asparagine, and Trp is an amino acid corresponding to a residue of tryptophan.

A gene encoding the polypeptide with the function of inhibiting Aβ42 protein aggregation mentioned above, a base sequence of the gene is CCACCAAAGA ACUGG, as shown in SEQ ID No:2 in the sequence listing, and a gene length is 15 bases;

wherein CCA is a codon of proline, AAG is a codon of lysine, AAC is a codon of asparagine, and UGG is a codon of tryptophan.

The polypeptide with the function of inhibiting Aβ42 protein aggregation according to the present invention can be synthesized by a polypeptide solid-phase synthesis method or a gene engineering technology;

wherein, when the polypeptide solid-phase synthesis method is used for synthesis, a standard Fmoc procedure is used, and a resin is selected from a 2-chlorotrityl chloride resin or a Wang resin; Fmoc is used to protect an N-terminal of an amino acid, and each protected amino acids are Fmoc-Pro-OH, Fmoc-Lys (boc)OH, Fmoc-Asn (trt)-OH and Fmoc-Trp (boc)-OH. The protected amino acids and the resin can be coupled one by one by using a coupling reagent and an activating reagent which are conventional in the field of solid-phase synthesis, which includes using 4-dimethylaminopyridine (DMAP) and dicyclohexylcarbodiimide (DCC) to complete the coupling of a first protected amino acid and the resin, and using 1-hydroxybenzotriazole (HOBt) to perform the coupling among the remaining protected amino acids. The protected amino acids and the resin are coupled one by one according to an amino acid sequence from a C-terminal to an N-terminal of the polypeptide, then the resin and a side chain protecting group of the protected amino acid are removed with a lysis buffer to obtain a crude product, and after purification of the crude product, the polypeptide with the function of inhibiting Aβ42 protein aggregation is obtained.

When the gene engineering technology is used for synthesis, an encoding gene is inserted into a vector, then the vector is transcribed into Escherichia coli of a prokaryotic expression system or yeast of a eukaryotic expression system for expression, and then a target polypeptide is separated and purified to obtain the polypeptide with the function of inhibiting Aβ42 protein aggregation.

Use of the polypeptide with the function of inhibiting Aβ42 protein aggregation includes use in preparation of a drug or a food resisting Aβ42 protein aggregation, and use in preparation of a drug, a health product or a food for preventing or treating Alzheimer's disease.

Compared with the prior art, the present invention has the following advantages and beneficial effects.

The polypeptide with the function of inhibiting Aβ42 protein aggregation according to the present invention can effectively inhibit aggregation and deposition of an Aβ42 protein, so as to effectively improve memory ability of a body; and can be applied to preparation of drugs or foods inhibiting Aβ42 protein aggregation or can be applied to preparation of a drug, a health product or a food for preventing or treating Alzheimer's disease, so that preventing and treating situations of the Alzheimer's disease can be effectively improved.

### BRIEF DESCRIPTION OF THE DRAWINGS

FIG. 1a is a high performance liquid chromatograph of a polypeptide with a function of inhibiting Aβ42 protein aggregation synthesized in Embodiment 1;

FIG. 1b is a mass spectrogram of a synthesized polypeptide with a function of inhibiting Aβ42 protein aggregation in Embodiment 1;

FIGS. 2a and 2b are respectively streaming images of HEK-293-mCherry cells (negative control group) and HEK-293-Aβ42-mCherry cells (model group) transfected with an Aβ42-red fluorescent labeling gene used in Embodiment 2;

FIGS. 3a and 3b are respectively IncuCyte Zoom long-time living cell images of the HEK-293-mCherry cells (negative control group) and the HEK-293-Aβ42-mCherry cells (model group) transfected with the Aβ42-red fluorescent labeling gene used in Embodiment 2;

FIGS. 4a and 4b are respectively IncuCyte Zoom long-time living cell images of intervention model groups with PW-5 at the concentrations of 0.05 mM and 0.5 mM in Embodiment 2;

FIG. 5 is a histogram of Aβ42 protein aggregation rates of the HEK-293-mCherry cells (negative control group), the HEK-293-Aβ42-mCherry cells (model group) transfected with the Aβ42-red fluorescent labeling gene, and intervention model groups with PW-5 at the concentrations of 0.05 mM and 0.5 mM in Embodiment 2; wherein ^{##} above the histogram indicates that compared with the model group, the negative control group has a significant statistical significance and p<0.01; and ** indicates that compared with the model group, the polypeptide PW-5 intervention groups have significant statistical significance and p<0.01.

### DETAILED DESCRIPTION OF THE PREFERRED EMBODIMENT

The technical solutions of the present invention are further described in detail below with reference to the specific embodiments and the accompanying drawings, but the embodiments and the scope of protection of the present invention are not limited hereto.

A polypeptide with a function of inhibiting Aβ42 protein aggregation according to the present invention is called PW-5, and has an amino acid sequence of Pro-Pro-Lys-Asn-Trp, as shown in sequence listing SEQ ID No:1;
wherein Pro is an amino acid corresponding to a residue of proline, Lys is an amino acid corresponding to a residue of lysine, Asn is an amino acid corresponding to a residue of asparagine, and Trp is an amino acid corresponding to a residue of tryptophan; and a molecular structural formula is as follows:

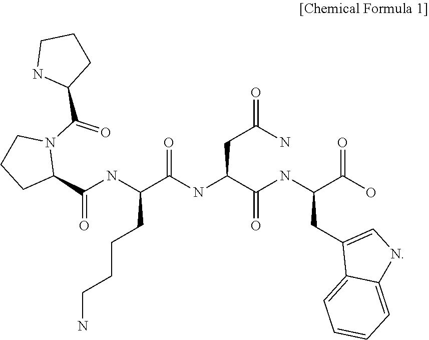

A gene encoding the polypeptide with the function of inhibiting Aβ42 protein aggregation mentioned above, a base sequence is CCACCAAAGAACUGG, as shown in sequence listing SEQ ID No:2, and a gene length is 15 bases;
wherein CCA is a codon of proline, AAG is a codon of lysine, AAC is a codon of asparagine, and UGG is a codon of tryptophan.

In the specific embodiments, in an application process of the polypeptide with the function of inhibiting Aβ42 protein aggregation according to the present invention, a mechanism of inhibiting AD senile plaque by the polypeptide is first researched through a visual in-vitro cell model test. Specifically, an amino acid sequence of a core component of senile plaque—an Aβ42 protein (the 22^{nd} amino acid in a sequence of 42 amino acids—glutamic acid is mutated into glycine) is stably transferred into HEK-293 cells, so that it is stably expressed in the HEK-293 cells to form a cell model of abnormal Aβ protein aggregation, and then tetracycline is added to induce the Aβ protein to form a red fluorescent protein and emit red fluorescent light for positioning and quantifying the expression of the Aβ42 protein; and then, the polypeptide with the function of inhibiting Aβ42 protein aggregation according to the present invention is added, and a HEK293 cell model transfected with an Aβ42-red fluorescent labeling gene is used for evaluation, thus proving that the polypeptide has a remarkable activity of inhibiting Aβ42 protein aggregation.

A HEK-293 cell strain is used in the visual in-vitro cell model to construct a cell model with stable expression of an exogenous gene Aβ42, which well simulates the characteristics of the core component of senile plaque of an AD patient, has better advantages than a conventional in-vitro cell model, is suitable for the research on senile plaque deposition of Alzheimer's disease, has very obvious advantages in screening a target food, drug acting on the senile plaque, and effectively and accurately positions the application of the polypeptide with the function of inhibiting Aβ42 protein aggregation.

### Embodiment 1

Synthesis of a Polypeptide with a Function of Inhibiting Aβ42 Protein Aggregation

1. Resin Selection

(1) A standard Fomc procedure was used, 0.0125 mmoL of 2-chlorotrityl chloride resin (GL Biochem (Shanghai) Ltd.) was selected at the beginning, 0.3 moL of a first Fmoc protected amino acid was added according to sequence characteristics of a C-terminal to an N-terminal of an amino acid sequence Pro-Pro-Lys-Asn-Trp, DCC and 5% (mass fraction) DMAP were added into a reactor for oscillating reaction, and the resin was washed with methylpyrrolidone (NMP) to remove excess protected amino acid;

(2) a standard Fomc procedure was used, 0.0125 mmoL of a Wang resin was selected at the beginning, 0.3 moL of a first Fmoc protected amino acid was added according to sequence characteristics of a C-terminal to an N-terminal of an amino acid sequence Pro-Pro-Lys-Asn-Trp, DCC and 5% (mass fraction) DMAP were added into a reactor for oscillating reaction, and the resin was washed with NMP to remove excess protected amino acid.

Coupling efficiency of the two resins is 94.34% for the 2-chlorotrityl chloride resin and 97.58% for the Wang resin, respectively.

2. Synthesis Process

A standard Fomc procedure was used, a Wang resin with a relatively high coupling efficiency was selected, a peptide chain was extended from a C-terminal to an N-terminal one by one according to sequence characteristics of an amino acid sequence Pro-Pro-Lys-Asn-Trp, a dosage of each amino acid was 0.1 moL, 0.4 moL of Fmoc protected amino acid was added, carboxyl of the protected amino acid was activated by adding HOBT in each condensation step, and 20% piperidine/DMF solution (15 mL/g) was used in each condensation step for processing for 20 minutes, so as to remove a Fmoc protecting group. After a peptide side chain was synthesized, a peptide chain containing resin was added into a mixed solution of dichloromethane and trifluoroacetic acid with a volume ratio of 99:1, and the peptide chain was cut off from the resin; and the polypeptide was added into a mixed solution of trifluoroacetic acid, ethylenediamine tartrate, distilled water and trypsin inhibitor (TIS) with a volume ratio of 94.5:2.5:2:1 for reaction for 2 hours, and a side chain protecting group was removed.

All the processes above were completed in a SYMPHONY-type 12-channel polypeptide synthesizer, the synthesized polypeptide was purified by a SHIMADZU high performance liquid chromatographic instrument to obtain a purity of more than 99%, and qualitative analysis was performed by a high performance liquid chromatograph (HPLC) and a mass spectrum (MS) to determine the amino acid sequence thereof.

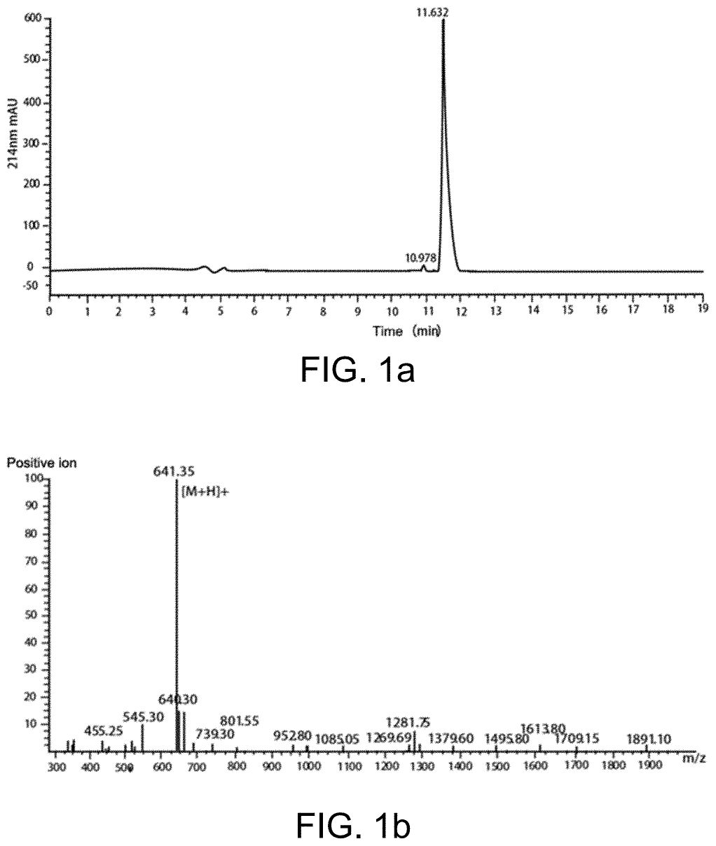

A high performance liquid chromatograph and a mass spectrogram of the synthesized polypeptide with the function of inhibiting Aβ42 protein aggregation are as shown in FIGS. 1a and 1b respectively, and it can be seen from FIGS. 1a and 1b as well as the analysis on the amino acid sequence that a primary amino acid sequence of the synthesized polypeptide is Pro-Pro-Lys-Asn-Trp, that is, a target polypeptide is obtained, and the polypeptide with the function of inhibiting Aβ42 protein aggregation is synthesized.

### Embodiment 2

Activity Experiment of Inhibiting Aβ42 Protein Aggregation In Vitro

1. Medium Preparation

High-glucose medium (DMEM), fetal bovine serum (FBS) and L-glutamine were prepared according to a ratio of 8.75:1:0.25 respectively; and meanwhile, 1 wt % double antibody (penicillin and streptomycin), 0.1 wt % Hygromycin B and 0.05 wt % of Blasticidin S antibiotic were added.

Preparation of 0.05 mM and 0.5 mM synthetic peptide (PW-5) solutions: 6.4 mg of synthetic peptide PW-5 was dissolved in 10 mL of medium, after the mixture was filtered with a 0.22 μm filter, a concentration of a stock liquor was 1 mM, and then the stock liquor was diluted to a concentration required by the experiment with the medium.

Preparation of 1 mg/mL tetracycline solution: 10 mg of tetracycline was weighed, 10 mL of PBS buffer was used for the preparation, and after the mixture was filtered with a 0.22 μm filter, the mixture was stored in a dark place at −20° C. for later use.

2. Observation of Aβ42 Protein Aggregation Model

HEK-293-mCherry cells (negative control) and HEK-293-Aβ42-mCherry cells (model) were cultured and experimented in a cell experiment.

A flow cytometry was used to detect the distribution of mCherry fluorescent proteins and whether a protein aggregation model was successful or not. Since the mCherry fluorescent proteins had an excitation wavelength of 580 nm and an emission wavelength of 610 nm, mCherry and protein aggregation expression in cells could be well detected and photographed by using the excitation wavelength corresponding to an Amnis imaging flow cytometer.

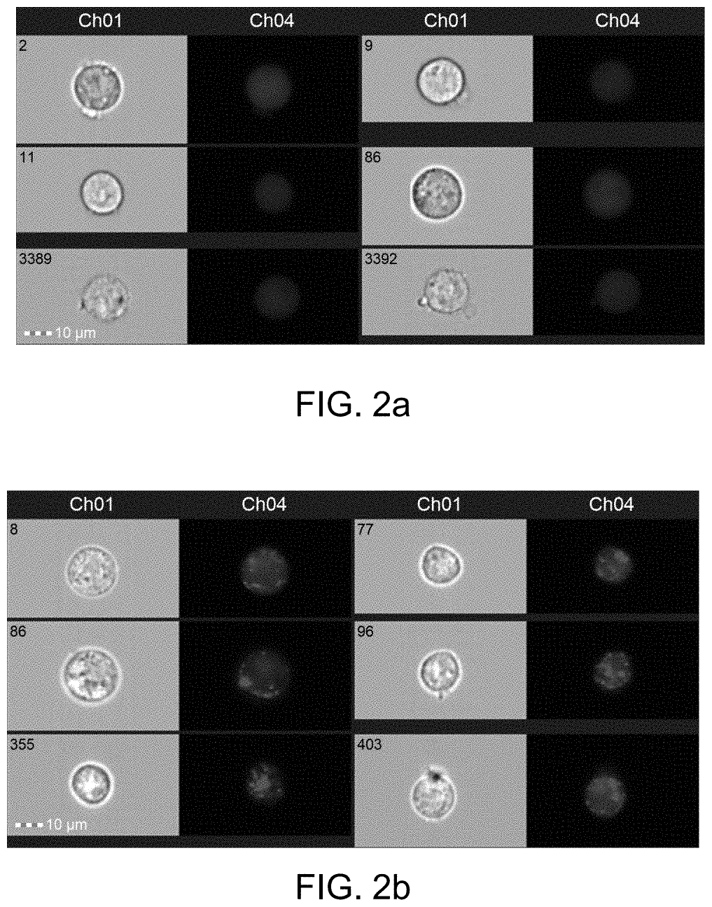

The negative control group (control group) refers to cells that do not contain the expression of the target protein Aβ42, while the model group refers to cells that express the target protein Aβ42 and are easy to aggregate. Streaming images of HEK-293-mCherry cells (negative control group) and HEK-293-Aβ42-mCherry cells (model group) transfected with an Aβ42-red fluorescent labeling gene are as shown in FIGS. 2a and 2b respectively, and it can be obviously seen from FIGS. 2a and 2b that no red fluorescent aggregation spot presents in the negative control group, and the cells are distributed with uniform red fluorescent background. However, multiple red fluorescent aggregation spots or blocks present in the cells of the model group, which indicates that the Aβ42 protein is successfully expressed, and the model group has significant difference compared with the negative control group.

3. Effect of Polypeptide on Aβ42 Protein Aggregation

The experimental groups were divided into a negative control group (control group, HEK-293-mCherry cells, without Aβ42 protein), a model group (model group, HEK-293-Aβ42-mCherry cells), and a PW-5 low-dose group (0.05 mM) and a PW-5 high-dose group (0.5 mM), and three parallels were set up in each group.

Cells were plated with a 24-well plate, a number of cells in each well were 5000, and after adherence for 24 hours, a medium and a polypeptide solution were added respectively according to the design experimental groups. After culturing for 48 hours, tetracycline (with a final concentration of 10 μg/mL) was added for induction, an IncuCyte Zoom long-time living cell imager was used for real-time tracking and photographing (the IncuCyte Zoom long-time living cell imager could be used to observe changes in a whole experimental process of cells in real time for a long time, which was better than an effect when Amnis was used to detect end-point protein aggregation), changes in protein aggregation in cells were observed after tetracycline was added, and the process was ended after continuing for 72 hours.

Double photographing of white light and fluorescent light was performed by an instrument, a magnification of photographing was 200 times, the photographing was performed once every 4 hours, in 9 fields of view for each well, a number of red fluorescent aggregation spots was observed, and a protein aggregation rate was calculated. The experiment was repeated three times and an average value of the results was taken.

Aβ42 protein aggregation rate (%)=(number of red fluorescent spots in field of view/number of cells in field of view)×100.

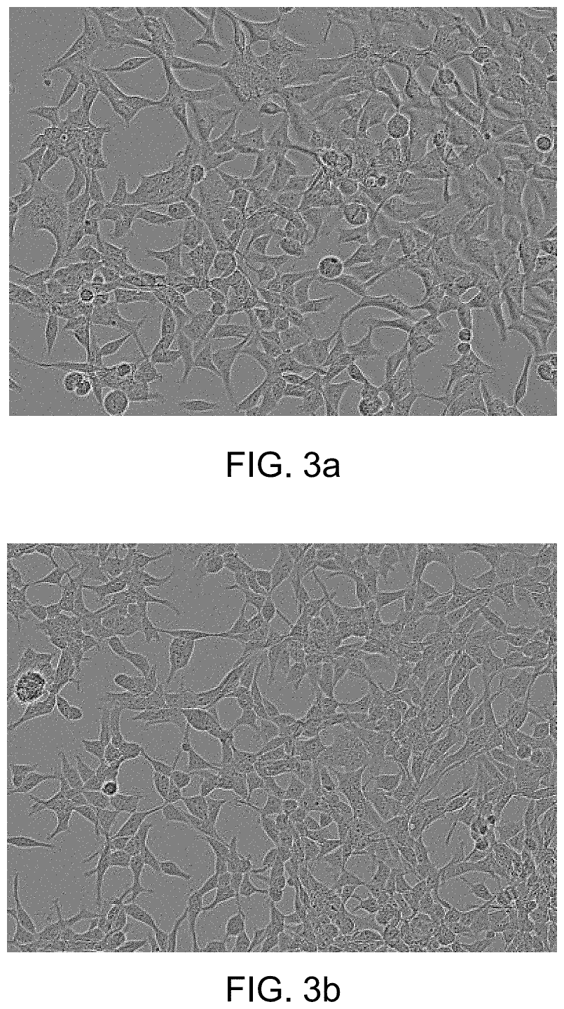

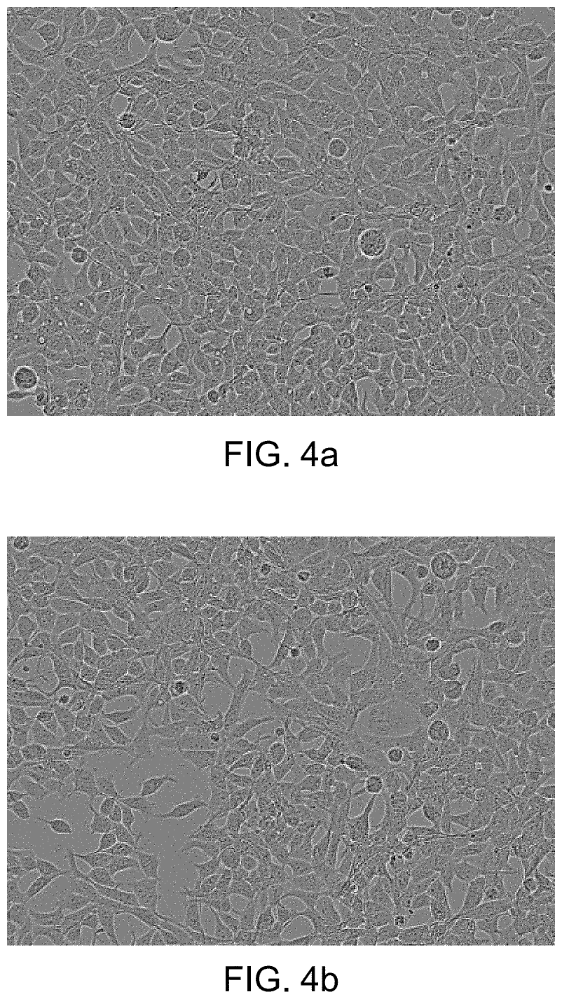

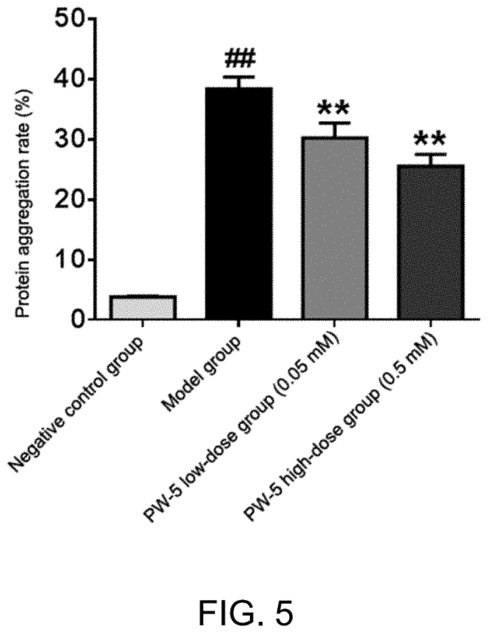

IncuCyte Zoom long-time living cell images of HEK-293-mCherry cells (negative control group), HEK-293-Aβ42-mCherry cells (model group) transfected with an Aβ42-red fluorescent labeling gene, and intervention model groups with PW-5 at the concentrations of 0.05 mM and 0.5 mM are as shown in FIGS. 3a, 3b, 4a and 4b respectively. A histogram of an Aβ42 protein aggregation rate of each experimental group is shown in FIG. 5. It can be seen from FIGS. 3a to 5 that after a low-dose group (0.05 mM) and a high-dose group (0.5 mM) of the synthesized polypeptide PW-5 interfere with a cell model, compared with the model group, red fluorescent aggregation spots of the Aβ42 protein in cells are significantly reduced, and the protein aggregation rate is significantly reduced. Moreover, an effect of the high-dose group is more significant than that of the low-dose group, showing a certain concentration dependent manner.

The results above show that the synthesized polypeptide according to the present invention has an obvious effect of inhibiting Aβ42 protein aggregation and an effect of improving memory, can be applied to the preparation of a drug or a food inhibiting Aβ42 protein aggregation, and can be applied to the preparation of a drug, a health product or a food for preventing or treating Alzheimer's disease.

The embodiments above are only the preferred implementation of the present invention and are only used to explain the present invention instead of limiting the present invention. Any changes, substitutions, combinations, simplifications and modifications made by those skilled in the art without departing from the spirit essence and the principle of the present invention shall be deemed as equivalent replacements and shall be included in the scope of protection of the present invention.

1. A polypeptide with a function of inhibiting Aβ42 protein aggregation, the polypeptide with the function of inhibiting Aβ42 protein aggregation in a name of PW-5, comprising an amino acid sequence of Pro-Pro-Lys-Asn-Trp, as shown in sequence listing SEQ ID No:1;
wherein Pro is an amino acid corresponding to a residue of proline, Lys is an amino acid corresponding to a residue of lysine, Asn is an amino acid corresponding to a residue of asparagine, and Trp is an amino acid corresponding to a residue of tryptophan.

2. A gene encoding the polypeptide with the function of inhibiting Aβ42 protein aggregation according to claim 1, comprising a base sequence of CCACCAAAGAACUGG, as shown in sequence listing SEQ ID No:2, and a gene length of 15 bases;
wherein CCA is a codon of proline, AAG is a codon of lysine, AAC is a codon of asparagine, and UGG is a codon of tryptophan.

3. Use of the polypeptide with the function of inhibiting Aβ42 protein aggregation according to claim 1, comprising a use in preparation of a drug or a food inhibiting Aβ42 protein aggregation, or a use in preparation of a drug, a health product or a food for preventing or treating Alzheimer's disease.


SEQUENCE LISTING (2 sequences)

SEQ ID NO: 1 - protein, 5 aa, Artificial Sequence
  Peptide
PPLAT

SEQ ID NO: 2 - RNA, 15 nt, Artificial Sequence
  Peptide
ccaccaaagaacugg